(19) United States
(12) Patent Application Publication
(10) Pub. No.: US 2021/0331949 A1
(43) Pub. Date: Oct. 28, 2021

(54) Automatic Electric Electronic Waste Handling Apparatus method and system

(71) Applicant: William Dean Hartman, Shasta Lake, CA (US)

(72) Inventor: William Dean Hartman, Shasta Lake, CA (US)

(21) Appl. No.: 16/858,620

(22) Filed: Apr. 25, 2020

(51) Int. Cl. C02F 1/50; C02F 1/68; C02F 1/00; G06Q 10/00; G06Q 50/06; G06Q 20/20; G06Q 30/04; G05B 15/02

(52) U.S. Cl. CPC C02F 1/50; C02F 1/686; C02F 1/008; G06Q 10/30; C02F 2103/002; G06Q 20/204; G06Q 20/209; G06Q 30/04; G05B 15/02; G06Q 50/06

(57) ABSTRACT

This present invention is a convenient automatic apparatus for handling sewage and grey-water which is tapable of removing waste from a waste holding tank, disinfecting a holding tank, and further allowing waste to be moved into a sewer or septic system through the use of a plurality of electric servo motors, sensors, cameras, hoses, booster motor, actuators and hydraulic system. This present invention can receive and transmit data via a wireless communication system, record information about the amount of waste product received and can communicate with the server software, generate a bill and generate a receipt. This present invention can allow a Recreational Vehicle, Autonomous Vehicle, Automobile, Travel Trailer, Airplane, Boat, Portable Toilet, Railroad Passenger Car, in a designated area, to connect the automatic electric electronic waste handling apparatus to the Release Mechanism affixed to a variety of vehicle holding tanks to remove waste from a black water, grey water holding tank, clean the holding tank and disinfect the holding tank. This present invention allows the vehicle to automatically dispose of waste matter, urine, feces, toilet paper and grey-water hands free in a manner that is safe for the environment and the end user, allowing for greater ease of use and efficiency as well as increased profits for businesses through labor savings.

### BACKGROUND

### 1. Field of the Invention

The invention relates generally to automatic electric electronic waste handling apparatus method and system

### 2. Background

The current process for waste removal from a holding tank requires many things. First, human interaction which is a major drawback due to the foul odor and potential contact with feces. Second, manually removing waste from a holding tank requires tools and technical know how. Third, in most cases the end user will have to dedicate a significant amount of valuable storage space in the vehicle, autonomous vehicle, airplane or boat space for the tools required to manually remove waste from a holding tank. In addition, the current method is difficult and can be a severe risk to health and the environment due to contact with waste matter or spillage of waste matter. Lastly, the current method requires an end user to locate personnel to allow access to a waste dump location. Other solutions attempt to make waste removal easy, but these solutions are similarly unable to meet the reeds of the industry because they fail to automate the process of waste removal from a holding tank and continue to risk health and environmental safety.

### SUMMARY OF THE INVENTION

It is desirable to have a device that hands free/automatically collects waste matter including urine, feces, toilet paper and grey-water from a Recreational Vehicle, Autonomous Vehicle, Travel Trailer, Airplane, Boat, Portable Toilet, Railroad Passenger Car, Module Building, Modular Office. Furthermore, it would also be desirable to have a device that cleans and disinfects a holding tank. Still, further, it would be desirable to have a device that communicates with a server software wirelessly to generate an invoice or bill and prints a receipt. Furthermore an apparatus that is able to detect the amount of Waste Matter in a Holding Tank. The disclosed device and associated method advantageously fill these needs and addresses the aforementioned deficiencies by providing an Automatic Electric Electronic Waste Handling Apparatus method and system.

Disclosed is a Automatic Electric Electronic Waste Handling Apparatus Method and System, which is made up of the following components Hydraulic Pump Motor, Hydraulic Hose Lines, Hydraulic Fittings, Booster Pump, Flexible Tubing, Vent, Telescoping Hydraulic Barrel and Piston, Angled Component Covers, Electric Motor, Electronic Digital Timer, motor drive shaft, rotation gear, Plurality of Pipes, Plurality of Pipe Fittings Software Server, wireless microcontroller module system for communication, Camera, a plurality of Sensors, a plurality of electronic servo motors, a plurality of actuators. Swivel Lift Plate, Release Mechanism, holding tank, chemical holding tank and measuring device. These components are connected as follows: The holding tank containing waste matter connects to the Release Mechanism allowing waste to flow through through the Release Mechanism and a measuring device, the waste then flows through a plurality of pipes and pipe fittings and into a sewer system, data from the measuring device is transmitted wirelessly then stored in a software server which is they able to generate an invoice or bill.

The device may also have one or more of the following: a screw lift or screw jack, wired communication, pipe handle, flow meter with number dial, power supply adapter. The screw lift or jack screw may be used manually or automatically, the pipe handler allow for the flexible pipe and fittings to be moved into a connected position, the flow meter with a number dial allow for visual confirmation of amount of waste collected, the power supply allows electricity to flow from the Automatic Electric Electronic Waste Handling Apparatus to the vehicle, airplane or travel trailer or in reverse form the vehicle to the Automatic Electric Electronic Waste Handling Apparatus. Similarly, the associated method may also include one or more of the following steps: Step 1 vehicle parks in designated area, Automatic Electric Electronic Waste Handling Apparatus is connected to holding tank located inside a vehicle or manufactured product. Step 2 Waste is then removed from a holding tank into the Automatic Electric Electronic Waste Handling Apparatus this may be done Automatically or Manually. Step 3 Waste is delivered into a sewer system. Step 4 the amount of waste accepted will be displayed on the flow meter number dial. Step 5 The flow meter number dial numbers displayed, the number dial may be electronic with a digital display or mechanical and will be sent to or given to the operator via a wired or wireless communication system, visually or through the communication server of the Automatic Electric Electronic Waste Handling Apparatus. The Automatic Electric Electronic Waste Handling Apparatus system will generate a bill or invoice.

The disclosed device is unique when compared with other known devices and solutions because it provides (1) An Automatic Electronic Waste Handling Apparatus to handle waste matter including feces, urine, toilet paper, grey-water (2) This present invention An Automatic Electric Electronic Waste Handling Apparatus communicates with the Release Mechanism, and (3) Can accurately measure the quantity of waste matter including feces, urine, toilet paper, grey-water. Similarly, the associated method is unique in that it: (1) Can operate automatically; (2) Can clean and disinfect the interior of a holding tank; and (3) Can supply to the holding tank an odor eliminating agent. Similarly, the disclosed method is unique when compared with other known processes and solutions in that it: (1) Communicates with a server; (2) Stores data; and (3) Creates an invoice or bill upon completion of service.

The disclosed device is unique in that it is structurally different from other known devices or solutions. More specifically, the device is unique due to the presence of (1) A Computerized Automatic device that automates the acceptance of waste matter, urine, feces, toilet paper, grey-water; (2) A Release Mechanism that consists of a wireless communication system and holding tank service system (3) An Automatic Device that consists f a plurality of electronic servo motors, pumos, server, flexible tubes, actuators, pipes and sensors contained in a system.

Furthermore, the process associated with the aforementioned device is likewise unique. More specifically, the disclosed process owes its uniqueness to the fact that the{1} Vehicle, Autonomous Vehicle, Travel Trailer, Airplane parks in designated area. {2} Vehicle, Autonomous Vehicle, Travel Trailer, Airplane emits signal to Waste Handling Apparatus to cue for service. {3} Waste Handling Apparatus leaves original position. {4} Waste Handling Apparatus is guided by a plurality of sensors, actuators, motors and cameras. {5} Waste Handling Apparatus connects to a Release Mechanism attached to a holding tank. {6} Waste Handling Apparatus presses into Pressure sensor to depress. {7} Waste Handling 4pparatus establishes positive connection. {8} Waste Handling Apparatus emits signal to Release Mechanism. {9} Valve opens allowing waste to leave holding tank. {10} Waste travels through measuring device. {11} Software records waste volume measurement. {12} Waste travels through Waste Handling Apparatus. {13} Waste enters into a sewer system. {14} Float Assembly emits signal when waste transfer is complete {15} Software communicates and cues holding tank cleaning. {16} Software communicates and engages chemical pump motor. {17} Chemical Pump Motor services Holding tank with cleaning chemicals, agents. {18} Software terminates cleaning cycle.{19} Release Mechanism closes valve. {20} Software communicates with Waste Handling Apparatus. {21} Software terminates Service to Vehicle, Autonomous Vehicle, Travel Trailer or Airplane. {22} Waste Handling Apparatus returns to original position. {23} Software stores Electronic Data in Server and communicates through a wireless device or wired device. {24} Software accesses Electronic Database for Pricing Matrix. {25} Software generates invoice or a bill for end user. (43) Software or Operator receive payment for service.

This disclosure will now provide a more detailed and specific description that will refer to the accompanying drawings. The drawings and specific descriptions of the drawings, as well as any specific or alternative embodiments discussed, are intended to be read in conjunction with the entirety of this disclosure. The Automatic electric electronic waste handling apparatus method and system may, however, be embodied in many different forms and should not be construed as being limited to the embodiments set forth herein; rather, these embodiments are provided by way of illustration only and so that this disclosure will be thorough, complete and fully convey understanding to those skilled in the art.

### BRIEF DESCRIPTION OF THE DRAWINGS

FIG. 1

View of automatic electric electronic waste handling apparatus & recreational vehicle

FIG. 2

Side end cutaway view automatic electric electronic waste handling apparatus

FIG. 3

Top View Cutaway of automatic electric electronic waste handling apparatus

FIG. 4

Front view of Release Mechanism

FIG. 5

Front view of Release Mechanism with valve partially open

FIG. 6

Side end cutaway view of automatic electric electronic waste handling apparatus

FIG. 7

View of automatic electronic door opening and closing mechanism

### DETAILED DESCRIPTION

The present invention is directed to automatic electric electronic waste handling apparatus method and system

In its most complete form, the device is made up of the following components Angled Component over (24), Hydraulic Pump (32), Hydraulic Lines (33), Hydraulic Fittings (34), Plurality of Hydraulic Telescoping Barrel and Pistons 18,19), Plurality of Flexible Tubing (21), Flexible Pipe (21),Flexible Tube Pipe Clamp (64) Swivel Lift Plate (17), Hydraulic Fluid Reservoir (35), Infrared Sensors (36), Optical Sensors (38), Unique Identification Aid (40), Magnetic Sensors (37), Pressure Sensor (16), A plurality or Wireless Communication Device (3), Server (42), Software (43), Waste Measuring Device (41), Chemical Pump Motor (25), Chemical Reservoir (45), Chemical Supply Lines (44), Chemical Blending and Dilution Device (55), Dispensing Nozzle (14), Waste Valve (2), Plurality of Pipes (21), Adjustable Elbow Pipe (47), Power Supply/Adapter (48), Power Charge Management System (49), Conductors/Wires (50), A Price Matrix (51), Electronic Float Assembly (53),Valve (2), Spring (8), Electronic Solenoid (5), Solenoid Securing Pin (6), Battery (4), Valve Retainer (7), Valve Retaining Pin (9), Liquid Service Port (10), Electrical Service Port (11), Ground Connection Port (12), Screw Threads (13), Threaded Screws (14), Release Mechanism Housing (1), Apparatus Bell Cover (28), Holding Tank (22), Electric Motor/Servo (30), Electric Motor Shaft and Gear (31), Rotation Gear (20), Electronic Servo (56), Controller (57), Amplifier (58), Control Circuit (59), Encode (60), Resolver (61), Leak Detector Device (54), Credit Card Reader (29), Payment Reader (29),Receipt Printer (63), Electronic Database (52). These components are connected as follows: This present invention Automatic Electric Electronic Waste Handling Apparatus is able to accept waste matter feces, urine, toilet paper, grey-water from a holding-tank, clean a holding tank and disinfect a holding tank automatically through the use of the Waste Handling Apparatus. It should further be noted that: The Automatic Electric Electronic Waste Handing Apparatus is able to accept waste matter feces, urine, toilet paper, grey-water, clean, disinfect a holding tank automatically through the use of a Hydraulic Powered or Pneumatic Powered device in connector with a plurality of Electric Motors, Chemicals, A plurality of Sensors, A Server, Software, Cameras, A Measuring Device, A plurality of Pipes, Flexible Tubes, Holding Tanks, A sewer connection, A Wireless Communication Device, in Connection with a Waste Handling Apparatus.

The most complete form of performing the method associated with the disclosed device consists of the following steps {1} Vehicle, Autonomous Vehicle, Travel Trailer, Airplane, boat parks in designated area. {2} Vehicle, Autonomous Vehicle, Travel Trailer, Airplane, boat emits signal to Automatic Electric Electronic Waste Handling Apparatus to cue for service. {3} Waste Handling Apparatus leaves original position. {4} Waste Handling Apparatus is guided by a plurality of sensors and camera. {5} Waste Handling Apparatus connects to a holding tank. {6} Waste Handling Apparatus presses into Pressure sensor to depress. {7} Waste Handling Apparatus establishes positive connection. {8} Waste Handling Apparatus emits signal to Release Mechanism. {9} Release Mechanism Valve opens allowing waste to leave holding tank. {10} Waste travels through measuring device. {11} Software records waste volume measurement. {12} Waste travels through Waste Handling Apparatus. {13} Waste enters into a holding tank or into a sewer system. {14} Float Assembly emits signal when waste transfer is complete. {15} Software communicates and cues holding tank cleaning. {16} Software communicates and engages chemical pump motor. {17} Chemical Pump Motor services Holding tank with cleaning chemicals, agents. {18} Software terminates cleaning cycle. {19} Release MEchanism closes valve. {20} Software communicates with Waste Handling Apparatus. {21} Software terminates Service to Vehicle, Autonomous Vehicle, Travel Trailer or Airplane {22} Waste Handling Apparatus returns to original position. {23} Software stores Electronic Data in Server and communicate.; through a wireless device or wired device. {24} Software accesses Electronic Database for Pricing Matrix. {25} Software generates invoice or a bill for end user. {43} Software or operator receive payment for service.

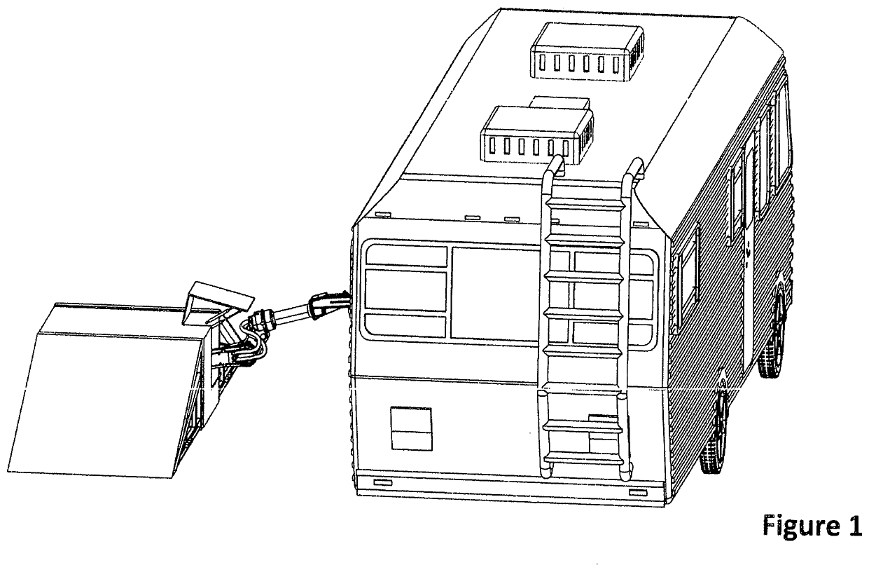

FIG. 1 Depicts cutaway view from the rear, a Recreational Vehicle (2.1) and a Automatic Electric Electronic Waste Handling Apparatus (1.1) connected to each other in a designated area. The vehicle pictured is a Recreational Vehicle (2.1) but this present invention the Automatic Electric Electronic Waste Handling Apparatus (1.1) is able to accept a automobile, autonomous vehicle, airplane, boat, travel trailer, train car, locomotive, modular house, modular office, portable toilet.

A automobile, autonomous vehicle, airplane, boat, travel trailer, train car, locomotive, modular house, modular office, portable toilet, containing a holding Tank (22) arrive at designated area. The Automatic Electric Electronic: Waste Handling Apparatus (1.1) connects to provide a service that removes waste matter, urine, feces, toilet paper and grey-water while simultaneously charging the Release Mechanism (1) and cleans the Holding Tank (22). The Automatic Electric Electronic Waste Handling Apparatus (1.1) will automatically attach to the Release Mechanism when a automobile, autonomous vehicle, airplane, boat, travel trailer, train car, locomotive, modular house, modular office, portable toilet enter the designated area creating a user free, efficient and safe way for removal of waste matter, urine, feces, toilet paper and grey-water. When the Automatic Electric Electronic Waste Handling Apparatus (1.1) has completed the service the software (43) will generate an invoice or bill. The Automatic Electric Electronic Waste Handling Apparatus (1.1) will disconnect from the Release Mechanism (1) and will return to the original position. The Release Mechanism (1) will close the Valve (2) not shown and will signal that the service is complete. Upon completion of service the Automatic Electric Electronic Waste Handling Apparatus (1.1) will generate a printed receipt via the Receipt Printer (63) and software (43) or can send an digital receipt via email.

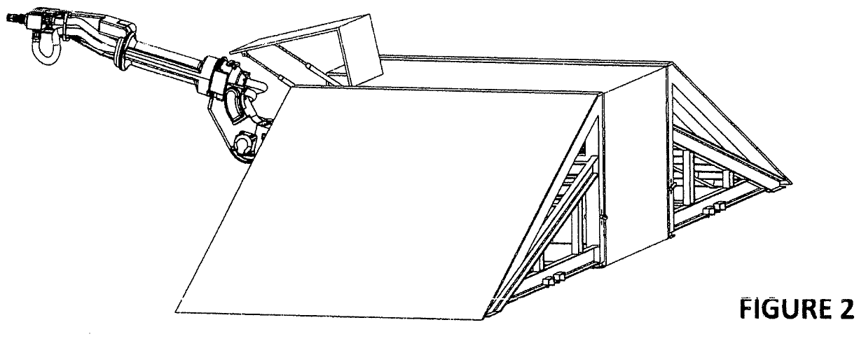

FIG. 2 depicts a cutaway view of the Automatic Electric Electronic Waste Handling Apparatus (1.1) connected to the Release Mechanism (1).

The Release Mechanism (1) enters into a designated area and emits a signal to the Automatic Electric Electronic Waste Handling Apparatus (1.1). The Automatic Electric Electronic Waste Handling Apparatus (1.1) cues service which will cause the Flexible Tube Pipe (21) to move into position via the Electric Motor (30), Electric Motor Shaft and Gear (31), Swivel Lift Plate (17) Flexible Pipe Tube (21) a plurality of Hydraulic Telescoping Piston (18) Hydraulic Telescoping Barrel (19) Hydraulic Pump (32) Hydraulic Lines (33) Hydraulic Fittings (34) guided by a plurality of Sensors and Cameras (36,37,38,39). The. Automatic Electric Electronic Waste Handling Apparatus (1.1) will establish a connection to the Release Mechanism (1) and will begin the service of removal of waste matter, urine, feces, toilet paper and grey-water, and will supply a charge to the Battery (4) via the Electrical Service Port (11), establish a Ground via the Ground Connector Port (12) and supply Liquid Cleaning Agent via the Liquid Service Port (10). The Automatic Electric Electronic Waste Handling Apparatus (1.1) will send and receive data to and from the Release Mechanism (1) via a Wireless Communication Device (3) Software (43), Server (42) Electronic Database (52).

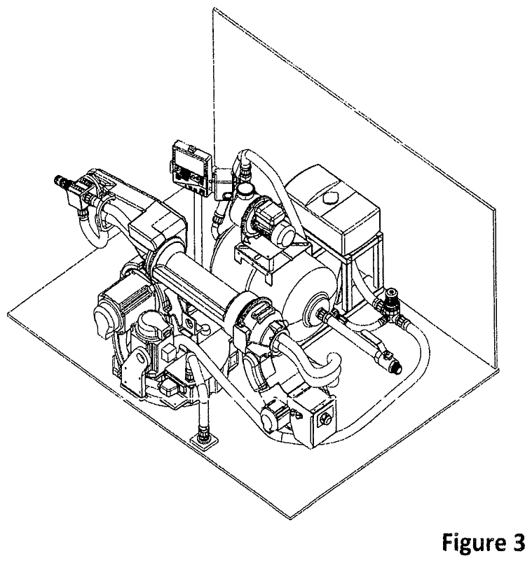

FIG. 3 depicts a top down cutaway view of the Automatic Electric Electronic Waste Handling Apparatus (1.1) and the components. The Automatic Electric Electronic Waste Handling Apparatus (1.1) is a counterpart device to the Release Mechanism (1). The Automatic Electric Electronic Waste Handling Apparatus (1.1) is the primary device that supplies power, cleaning agents, removes waste matter, urine, feces, toilet paper and grey-water from a Holding Tank (22) and accepts that waste matter, urine, feces, toilet paper and grey-water into a Holding Tank or sewer system.

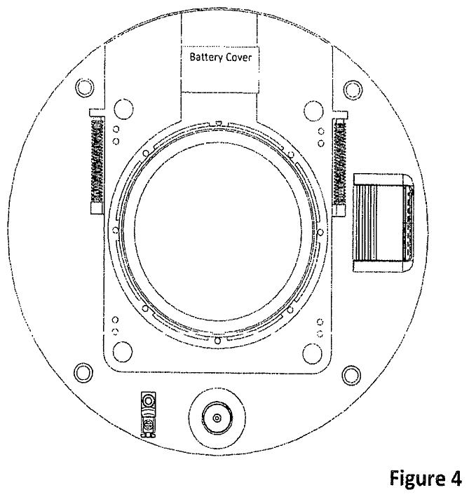

FIG. 4 depicts The Release Mechanism (1) which attaches to a Holding Tank (22) once the Release Mechanism is attached to the Holding Tank (22) not shown, the Release Mechanism (1) is able to receive information from the Electronic Float Assembly (53) not shown, about the amount of waste matter in the Holding Tank (22). When a vehicle is in a designated area the Release Mechanism (1) begins service by emitting a signal to cue the Automatic Electric Electronic Waste Handling Apparatus (1.1) not shown via a Wireless Communication Device (3). The Automatic Electric Electronic Waste Handling Apparatus (1.1) leaves original position and connects to the Release Mechanism (1) simultaneously connecting the Liquid Service Port (10), the Electrical Service Port (11), the Ground Connection Port (12), the Flexible Tube Pipe (21) and depresses the Pressure Sensor (16) Once a positive connection is made between the Automatic Electric Electronic Waste Handling Apparatus (1.1) and the Release Mechanism (1) the Release Mechanism (1) will cue the Electronic Solenoid (5) which will retract the Solenoid Securing Pin (6) which will allow the Valve (2) to open by means of a spring (8) which will allow waste matter, urine, feces, grey-water to flow through the Release Mechanism (1) into the Automatic Electric Electronic Waste Handling Apparatus (1.1) not shown and into a Flexible Tube Pipe (21) not shown. The waste matter, urine, feces, toilet paper and grey-water will travel through the Flexible Tube Pipe (21) not shown past a Measuring Device (41) not shown into a Holding Tank (22) not shown or into a Sewer System. When the Measuring Device (41) not shown no longer receives data about passing waste matter the Automatic Electric Electronic Waste Handling Apparatus (1.1) will emit a signal to the Release Mechanism (1) to close the Valve (2) by way of a Electronic Servo Motor (56). The Automatic Electric Electronic Waste Handling Apparatus (1.1) will send a signal to cue the Chemical Pump Motor (25) not shown which will cause liquid cleaning agents to travel through the Flexible Tube Pipe (21) not shown, and through the Release Mechanism (1) via the Liquid Service Port (10), through the Dispensing Nozzle (14) not shown and into a Holding Tank (22) not shown to clean and treat the Holding Tank (22). Simultaneously the Automatic Electric Electronic Waste Handling Apparatus (1.1) charges the Battery (4) located inside the Release Mechanism (1). The Release Mechanism (1) establishes a ground through the Ground Connection Port (12) through the Automatic Electric Electronic Waste Handling Apparatus (1.1) not shown, and to the Ground (62) not shown. When the service is complete the Automatic Electric Electronic Waste Handling Apparatus (1.1)

FIG. 4 depicts an embodiment of the Release Mechanism (1)and its components, Valve (2), Wireless Communication Device (3), Battery (4), Electronic Solenoid (5), Solenoid Securing Pin (6), Valve Retainer (7), Spring (8), Valve Retaining Pin (9), Liquid Service Port (10), Electrical Service Port (11), Ground Connection Port (12), Threaded Screw (15), Pressure Sensor (16), Electronic Servo Motor (56).

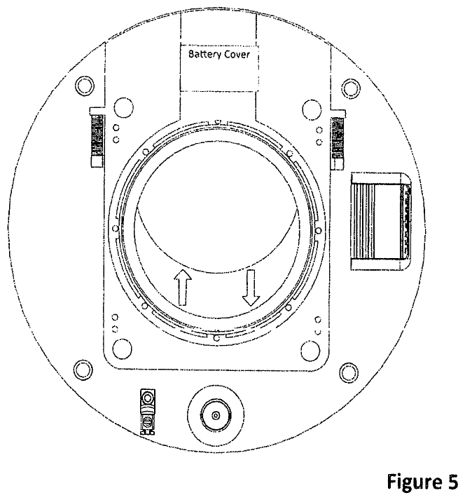

FIG. 5 Depicts The Release Mechanism (1) attached to a Holding Tank (22) once the Release Mechanism is attached to the Holding Tank (22), the Release Mechanism (1) is able to receive information from the Float Assembly (53), about the amount of waste matter in the Holding Tank (22). When a vehicle is in a designated area the Release Mechanism (1) begins service by emitting a signal to cue the Automatic Electric Electronic Waste Handling Apparatus (1.1) not shown via a Wireless Communication Device (3). The Automatic Electric Electronic Waste Handling Apparatus (1.1) leaves original position and connects to the Release Mechanism (1) simultaneously connecting the Liquid Service Port (10), the Electrical Service Port (11), the C round Connection Port (12), the Flexible Tube Pipe (21) and depresses the Pressure Sensor (16) Once a connection is made between the Automatic Electric Electronic Waste Handling Apparatus (1.1) and Release Mechanism (1) the Release Mechanism (1) will cue the Electronic Solenoid (3) which will retract the Solenoid Securing Pin (6) which will allow the Valve (2) to open by means of a spring (8) which will allow waste matter, urine, feces, grey-water to flow through the Release Mechanism (1) into the Automatic Electric Electronic Waste Handling Apparatus (1.1) not shown and into a Flexible Tube Pipe (21). The waste matter, urine, feces, toilet paper and grey-water will travel through the Flexible Tube Pipe (21) past a Measuring Device (41) not shown into a Holding Tank (22) or into a Sewer System. When the Measuring Device (41) not shown no longer receives data about passing waste matter the Automatic Electric Electronic Waste Handling Apparatus. (1.1) will emit a signal to the Release Mechanism (1) to close the Valve (2) by way of a Electronic Servo Motor (56). The Automatic Electric Electronic Waste Handling Apparatus (1.1) will send a signal to cue the Chemical Pump Motor (25) not shown which will cause liquid cleaning agents to travel through the Flexible Tube Pipe (21) not shown, and through the Release Mechanism (1) via the Liquid Service Port (10), through the Dispensing Nozzle (14) and into a Holding Tank (22) to clean and treat the Holding Tank (22).Simultaneously the Automatic Electric Electronic Waste Handling Apparatus (1.1) charges the Battery (4) located inside the Release Mechanism (1). The Release Mechanism (1) establishes a ground through the Ground Connection Port (12) through the Automatic Electric Electronic Waste Handling Apparatus (1.1) not shown, and to the Ground (62) not shown. When the service is complete. the Automatic Electric Electronic Waste Handling Apparatus (1.1) will return to original position and generate a receipt via receipt printer (63) or can electronically send an email receipt.

FIG. 5 depicts a cutaway side view, embodiment of the Release Mechanism (1) and its components, Valve (2), Wireless Communication Device (3), Battery (4), Electronic Solenoid (5), Solenoid Securing Pin (6), Valve Retainer (7), Spring (8), Valve Retaining Pin (9), Liquid Service Port (10), Electrical Service Port (11), Ground Connection Port (12) not shown, Threaded Screw (15), Pressure Sensor (16), Electronic Servo Motor (56). Shown in FIG. 5 Flexible Tube Pipe (21) Holding Tank (22), Float Assembly (53).

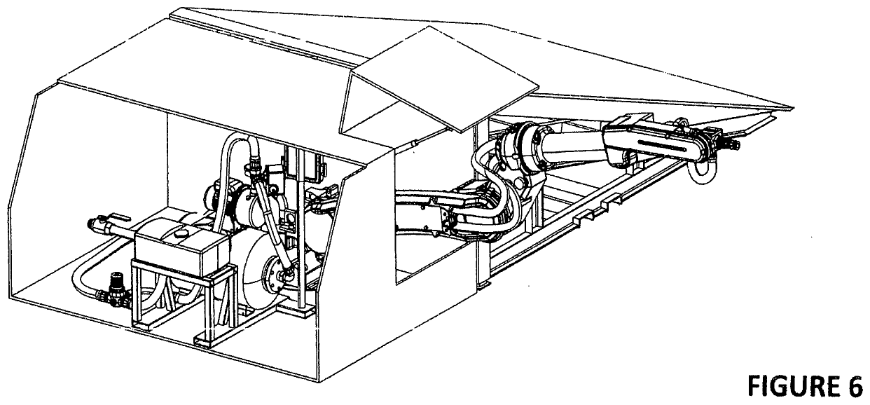

FIG. 6 depicts a cutaway side view of the Automatic Electric Electronic Waste Handling Apparatus (1.1). Embodied in this illustration is a power supply/adapter (48), Angled Component Cover (24) Conductors/Wires (50), Electric Motor Shaft and Gear (31), Flexible Tube Pipe (21), The Automatic Electric Electronic Waste Handling Apparatus (1.1) is supplied with AC or DC power which is used to power an Electric Motor (30) riot shown, a Hydraulic Pump (32) not shown, a Chemical Pump Motor (25) not shown, a Wireless Communication Device (3) not shown. The Electric Motor (30) rotates in a forward and reverse direction the Electric Motor Shaft and Gear (31) not shown, then rotates the Rotation Gear (20) not shown in a forward or reverse direction. The Rotation Gear (20) not shown is connected to the Flexible Pipe Tube (21). The Angled Component Cover (24) provides protection to the Automatic Electric Electronic Waste Handing Apparatus Components in the even a vehicle drives onto the Angled Component Cover (24).

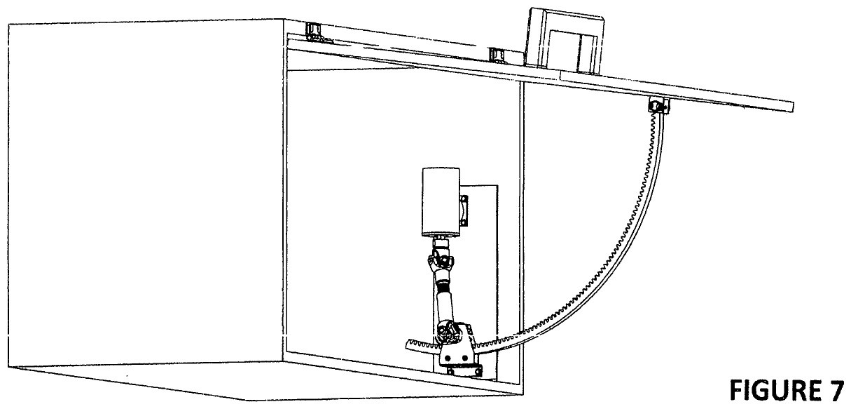

FIG. 7 Depicts a Recreational Vehicle (2.1) and a Automatic Electric Electronic Waste Handling Apparatus (1.1) connected to each other in a designated area. The vehicle pictured is a Recreational Vehicle (2.1) but this present invention the Automatic Electric Electronic Waste Handling Apparatus (1.1) is able to accept a automobile, autonomous vehicle, airplane, boat, travel trailer, train car, locomotive, modular house, modular office, portable toilet, automobile, autonomous vehicle, airplane, boat, travel trailer, train car, locomotive, modular house, modular office, portable toilet. containing a holding Tank (22) not shown arrive at designated area. The Automatic Electric Electronic Waste Handling Apparatus (1.1) connects to provide a service that removes waste matter, urine, feces, toilet paper and grey-water while simultaneously charging the Release Mechanism (1) and cleans the Holding Tank (22) not shown. The Automatic Electric Electronic Waste Handling Apparatus (1.1) will automatically attach to the Release Mechanism when a automobile, autonomous vehicle, airplane, boat, travel trailer, train car, locomotive, modular house, modular office, portable toilet enter the designated area creating a user free, efficient and safe way for removal of waste matter, urine, feces. toilet paper and grey-water. When the Automatic Electric Electronic Waste Handling Apparatus (1.1) has completed the service the software (43) will generate an invoice or bill. The Automatic Electric Electronic Waste Handling Apparatus (1.1) will disconnect from the Release Mechanism (1) and will return to the original position. The Release Mechanism (1) will close the Valve (2) and will signal that the service is complete.

FIG. 8 Depicts a view as seen from the front or rear of the Automatic Electric Electronic Waste Handling Apparatus (1.1) connected to the Release Mechanism (1), the Release Mechanism (2) connected to a Holding Tank (22), the Holding Tank (22) containing a Electronic Float Assembly (53) not shown. FIG. 8 depicts the Flexible Tube Pipe (21), a plurality of Hydraulic Telescoping Barrel (19), a plurality of Hydraulic Telescoping Piston (18), a plurality of Rotation Gear (20) a plurality of Flexible Tube Pipe Clamp (64), a plurality of Electronic Servo Motor (56), a plurality of Adjustable Elbow (47). The Automatic Electric Electronic Waste Handling Apparatus (1.1) receives a signal from the connected to the Electronic Waste Handling Connection Device (1), and cues service. The Automatic Electric Electronic Waste Handling Apparatus (1.1) leaves the original position, connects to the Release Mechanism (1)guided by a plurality of Sensors (36)(37)(38), not shown Camera (39), not shown Software (42) Server not shown (43) not shown and Wireless Communication Device (3) not shown, connected by the Flexible Pipe Tube (21) which is maneuvered by a plurality of Hydraulic Telescoping Barrel (19) Hydraulic Telescoping Piston (18) Electronic Servo Motor (56), Rotation Gear and Adjustable Elbow (47) and Swivel Lift Plate (17). The Flexible Tube Pipe is connected to the Hydraulic Telescoping Barrel (19) via the Flexible Tube Pipe Clamp (64) and begins the cued service. When the waste matter, urine, feces, toilet paper and grey-water has been received from the Holding Tank (22) and the Holding Tank (22) is cleaned via the Liquid Service Port (10), the Battery (4) not shown is charged via the Electrical Service Port (11) the Automatic Electric Electronic Waste Handling Apparatus (1.1) will disconnect from the Release Mechanism (1) and will return to the original position. Upon comoletion of service the software (43) will generate an invoice or bill which can be paid through the Payment Reader Card Reader (29) and will print a receipt after payment is received via the receipt printer (63).

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to be understood that this disclosure is not limited to any single specific embodiments or enumerated variations. Many modifications, variations and other embodiments will come to mind of those skilled in the art, ant which are intended to be and are in fact covered by this disclosure. It is indeed intended that the scope of this disclosure should be determined by a proper legal interpretation and construction of the disclosure, including equivalents, as understood by those of skill in the art relying upon the complete disclosure present at the time of filing.

What is claimed is:

1. An automatic electric electronic waste handling apparatus for draining and cleaning holding tanks comprised of the following parts:
a) sensors for guiding the automatic electric electronic waste handling apparatus into a connecting position at holding tanks in vehicles;
b) a release fitting mechanism affixed to sewage and grey water holding tanks;
c) a swivel plate for lifting the flex pipe to meet the release fitting mechanism.
d) a measuring device for determining the amount of sewage and grey water removed from the holding tank;
e) flexible pipes for transferring waste from the holding tank to sewer;
f) a plurality of sensors for determining capacities;
g) an automatic electric electronic waste handling apparatus water holding tank;
h) a chemical reservoir for holding cleaning solution;
i) a pump for dispensing cleaning solution;
j) a payment reader for accepting payments;
k) a software application for monitoring draining and cleaning operations;
l) a server for processing payments and recording operations;
m) a wireless communication system device for communicating with the server and customer;
n) a plurality of actuators, hoses, servo motors for transferring waste from holding tank to sewer through service ports; and
o) a controller for managing onboard operations.

2. The automatic electric electronic waste handling apparatus for draining and cleaning holding tanks of claim 1 wherein the release mechanism includes a waste valve, actuator, infrared sensors, electronic digital timer, power source and a pressure sensor.

3. The automatic electric electronic waste handling apparatus for draining and cleaning holding tanks of claim 1 wherein the measuring device includes a flow meter, a number dial and a digital display.

4. The automatic electric electronic waste handling apparatus for draining and cleaning holding tanks of claim 1 wherein the flexible pipes include flexible tubing, and flexible pipe clamps.

5. The automatic electric electronic waste handling apparatus for draining and cleaning holding tanks of claim 1 wherein the holding tank includes a leak detection device, a float assembly and a swivel lift plate.

6. The automatic electric electronic waste handling apparatus for draining and cleaning holding tanks of claim 1 wherein the chemical reservoir includes a chemical blending and dilution device.

7. The automatic electric electronic waste handling apparatus for draining and cleaning holding tanks of claim 1 wherein the hydraulic pump includes hydraulic fittings, a hydraulic telescoping barrel and piston and, hydraulic or pneumatic drives.

8. The automatic electric electronic waste handling apparatus for draining and cleaning holding tanks of claim 1 wherein the payment reader includes a card reader and a digital receipt and invoice generator.

9. The automatic electric electronic waste handling apparatus for draining and cleaning holding tanks of claim 1 wherein the software includes waste volume recording.

10. The automatic electric electronic waste handling apparatus for draining and cleaning holding tanks of claim 1 wherein the server includes a price matrix and billing statement operations.

11. The automatic electric electronic waste handling apparatus for draining and cleaning holding tanks of claim 1 wherein the controller includes an amplifier, cue signals, an encoder, and a resolver.

12. A method of draining and cleaning sewage and grey water holding tanks. The method is comprised of the following steps:
a) emitting a service signal cue;
b) guiding the automatic electric electronic waste handling apparatus into a connecting position to holding tanks;
c) depressing pressure sensor;
d) communicating with affixed release fitting mechanism to vehicle holding tank;
e) emitting a signal from the release fitting mechanism to the automatic electric electronic waste handling apparatus;
f) opening the waste valve;
g) draining sewage and grey water holding tank;
h) measuring waste leaving sewage and grey water holding tank;
i) emitting a signal from the EDM float assembly to the automatic electric electronic waste handling apparatus;
j) engaging chemical pump and booster motor;
k) transferring disinfectant chemical blend into sewage and grey water holding tank;
l) closing the waste valve;
m) transferring chemical from sewage and grey water holding tank into the automatic electric electronic waste handling apparatus;
n) transferring contents of the automatic electric electronic waste handling apparatus into the sewer;
o) storing electronic data in server;
p) accessing electronic database for pricing matrix; and
q) completing a financial transaction.

13. The method of draining and cleaning sewage and grey water holding tanks of claim 14 wherein emitting a service signal cue includes the step the Release Mechanism or user sending the signal due to the automatic electric electronic waste handling apparatus using the software of claim 1.

14. The method of draining and cleaning sewage and grey water holding tanks of claim 14 wherein the guiding the automatic electric electronic waste handling apparatus into a connecting position to holding tanks includes the step of the sensors of claim 1 determining distances to the vehicle holding tank.

15. The method of draining and cleaning sewage and grey water holding tanks of claim 14 wherein depressing pressure sensor includes the step of the pressure sensor being depressed when the release fitting mechanism makes contact with the electric electronic waste handling apparatus.

16. The method of draining and cleaning sewage and grey water holding tanks of claim 14 wherein the measuring waste leaving vehicle holding tank includes the step of calculating the volume of waste and sending volume data to the server and into the pricing matrix.

17. The method of draining and cleaning sewage and grey water holding tanks of claim 14 wherein the engaging chemical pump motor also includes the step of mixing the chemicals.

18. The method of draining and cleaning sewage and grey water holding tank of claim 14 wherein the engaging of a booster pump motor includes the step of mixing water with chemicals.

19. The method of draining and cleaning sewage and grey water holding tanks of claim 14 wherein the completing a financial transaction includes the step of allowing the user to make a payment and receive a receipt.
